(19) United States
(12) Patent Application Publication
(10) Pub. No.: US 2017/0294628 A1
(43) Pub. Date: Oct. 12, 2017

(54) Organic Light Emitting Diode and Light Emitting Diode Display

(71) Applicant: Samsung Display Co., Ltd., Yongin-Si (KR)

(72) Inventors: Dong Hoon Kim, Sunwon-si (KR); Sang Hoon Yim, Suwon-si (KR); Kwan Hee Lee, Suwon-si (KR); Myeong Suk Kim, Hwaseong-si (KR); Sung Wook Kim, Hwaseong-si (KR); Chang Woong Chu, Hwaseong-si (KR); Jin Soo Hwang, Seoul (KR)

(21) Appl. No.: 15/481,082

(22) Filed: Apr. 6, 2017

(30) Foreign Application Priority Data
Apr. 7, 2016 (KR) 10-2016-0042916
Apr. 4, 2017 (KR) 10-2017-0043933

(51) Int. Cl. H01L 51/52; H01L 51/00; H01L 27/32; C07C 211/58; C07D 209/86; C07D 307/91; C07C 211/54; C07D 333/76; H01L 51/50; C09K 11/06

(52) U.S. Cl. CPC H01L 51/5284; H01L 51/5206; H01L 51/5221; H01L 51/504; H01L 51/0097; H01L 51/5253; H01L 27/3262; C09K 11/06; H01L 51/0061; H01L 51/0059; H01L 51/006; C07D 209/86; C07D 307/91; C07C 211/54; C07D 333/76; C07C 211/58; H01L 2251/5338; H01L 51/0072; H01L 51/0073; H01L 51/0052; H01L 51/0074; H01L 51/0058; C07C 2603/18

(57) ABSTRACT

A light emitting diode according to an exemplary embodiment of the present invention includes: a first electrode; a second electrode overlapping the first electrode; an emission layer disposed between the first electrode and the second electrode; and a capping layer disposed on the second electrode, wherein the capping layer satisfies Equation 1 below.

n*k(λ=405 nm)≦0.8. Equation 1

In Equation 1, n*k (λ=405 nm) represents an optical value that is a product of a refractive index and an absorption coefficient in a 405 nanometer wavelength.

### CROSS-REFERENCE TO RELATED APPLICATION

This application claims priority to and the benefit of Korean Patent Application No. 10-2016-0042916 filed in the Korean Intellectual Property Office on Apr. 7, 2016 and No. 10-2017-0043933 filed in the Korean Intellectual Property Office on Apr. 4, 2017, the entire contents of which are incorporated herein by reference.

### BACKGROUND

(a) Field

The disclosure relates to an organic light emitting diode and a light emitting diode display, and more specifically, relates to an organic light emitting diode that perceives minimal damage from radiation of a light having a harmful wavelength and a light emitting diode display.

(b) Description of the Related Art

Recently, display devices including an organic light emitting diode has become increasingly popular. As more people use display devices that incorporate organic light emitting diode, the display devices becomes used in a wider range of environments than before.

However, in the display device including the organic light emitting diode, the organic emission layer is easily damaged by elements in the environment. This results in an undesirably short product life span. There is a need for a display device that is usable in various environments and offers excellent light efficiency without being so vulnerable to environmental elements.

The above information disclosed in this Background section is only for enhancement of understanding of the background of the invention and therefore it may contain information that does not form the prior art that is already known in this country to a person of ordinary skill in the art.

### SUMMARY

Exemplary embodiments provide an organic light emitting diode and a light emitting diode display that are prevented from being degraded by a light having a harmful wavelength.

However, objects to be solved by the embodiments of the present invention are not limited to the above-mentioned problems and can be variously extended within the scope of the technical idea included in the present invention.

A light emitting diode according to an exemplary embodiment of the present invention includes a first electrode; a second electrode overlapping the first electrode; an emission layer disposed between the first electrode and the second electrode; and a capping layer disposed on the second electrode, wherein the capping layer satisfies Equation 1 below.

n*k(λ=405 nm)≧0.8 Equation 1

In Equation 1, n*k (λ=405 nm) represents an optical value that is a product of a refractive index and an absorption coefficient in a 405 nanometer wavelength.

A light emitting diode display according to an exemplary embodiment of the present invention includes: a substrate; a transistor disposed on the substrate; a light emitting diode connected to the transistor; and an encapsulation layer disposed on the light emitting diode, wherein the light emitting diode includes a first electrode, a second electrode overlapping the first electrode, an emission layer disposed between the first electrode and the second electrode, and a capping layer disposed on the second electrode, and the capping layer satisfies Equation 1 below.

n*k(λ=405 nm)≧0.8 Equation 1

In Equation 1, n*k (λ=405 nm) represents an optical value that is a product of a refractive index and an absorption coefficient in a 405 nanometer wavelength.

An organic light emitting diode according to an exemplary embodiment includes: a first electrode; a second electrode overlapping the first electrode; an organic emission layer disposed between the first electrode and the second electrode; and a capping layer disposed on the second electrode, wherein the capping layer has an absorption rate of 0.25 or more in a 405 nanometer wavelength, and the capping layer includes at least one among materials represented by Chemical Formula A-1 to Chemical Formula A-3 and Chemical Formula B-1.

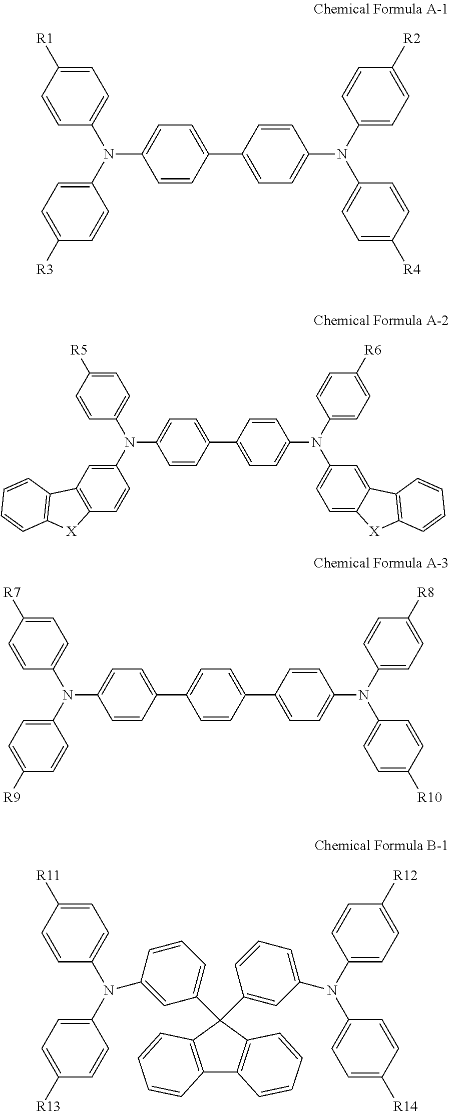

In Chemical Formula A-1 to Chemical Formula A-3, R1 to R10 are independently one of hydrogen, an alkyl group having 1 to 3 carbon atoms, a phenyl group, a carbazole group, a dibenzothiophene group, a dibenzofuran group, and a biphenyl group, and X is one of an oxygen atom, a sulfur atom, and a nitrogen atom, while in Chemical Formula B-1, R11 to R14 are independently one of hydrogen, an alkyl group having 1 to 3 carbon atoms, a phenyl group, a carbazole group, a dibenzothiophene group, a dibenzofuran group, and a biphenyl group.

According to exemplary embodiments, as the light of the harmful wavelength region is blocked, the degradation of the organic emission layer may be prevented, and the organic light emitting diode of which the blue emission efficiency is not inhibited may be provided.

Also, the light emitting diode display having the flexible substrate of which the lifespan increases may be provided.

### BRIEF DESCRIPTION OF THE DRAWINGS

FIG. 1 is a view schematically showing a structure of an organic light emitting diode according to an exemplary embodiment of the described technology.

FIG. 2 is a view schematically showing a structure of an organic light emitting diode according to another exemplary embodiment of the described technology.

FIG. 3 is a graph showing an absorption rate, a refractive index, transmittance, and a sunlight spectrum of a capping layer material corresponding to Exemplary Embodiment 1.

FIG. 4 is a graph showing an absorption rate, a refractive index, transmittance, and a sunlight spectrum of a capping layer material corresponding to Comparative Example 1.

FIG. 5 is a cross-sectional view schematically showing a light emitting diode according to an exemplary embodiment of the described technology.

FIG. 6 is a graph showing a relation of an optical value (a product of a refractive index and an absorption coefficient) and a transmittance according to exemplary embodiment of the described technology.

FIG. 7 is a graph showing optical constants of a capping layer according to a comparative example.

FIG. 8 is a graph showing a relation of an optical value (a product of a refractive index and an absorption coefficient) and a blue emission efficiency decreasing value according to an exemplary embodiment of the described technology.

FIG. 9 is a cross-sectional view of a light emitting diode display according to an exemplary embodiment of the described technology.

### DETAILED DESCRIPTION OF THE EMBODIMENTS

The described technology will be described more fully hereinafter with reference to the accompanying drawings, in which exemplary embodiments are shown. As those skilled in the art would realize, the described embodiments may be modified in various different ways, all without departing from the spirit or scope of the described technology.

In order to clearly explain the described technology, aspects or portions that are not directly related to the described technology are omitted, and the same reference numerals are attached to the same or similar constituent elements throughout the entire specification.

In addition, the size and thickness of each configuration shown in the drawings are arbitrarily shown for better understanding and ease of description, and the described technology is not limited thereto. In the drawings, the thickness of layers, films, panels, regions, etc., are exaggerated for clarity. In the drawings, for better understanding and ease of description, the thicknesses of some layers and areas may be exaggerated.

It will be understood that when an element such as a layer, film, region, or substrate is referred to as being “on” another element, it can be directly on the other element or intervening elements may be present. In contrast, when an element is referred to as being “directly on” another element, there are no intervening elements present. Further, in the specification, the word “on” or “above” means disposed on or below the object portion, and does not necessarily mean disposed on the upper side of the object portion based on a gravitational direction.

In addition, unless explicitly described to the contrary, the word “comprise” and variations such as “comprises” or “comprising” will be understood to imply the inclusion of stated elements but not the exclusion of any other elements.

Further, in the specification, the phrase “on a plane” means viewing the object portion from the top, and the phrase “on a cross-section” means viewing a portion of the object that is vertically cut from the side.

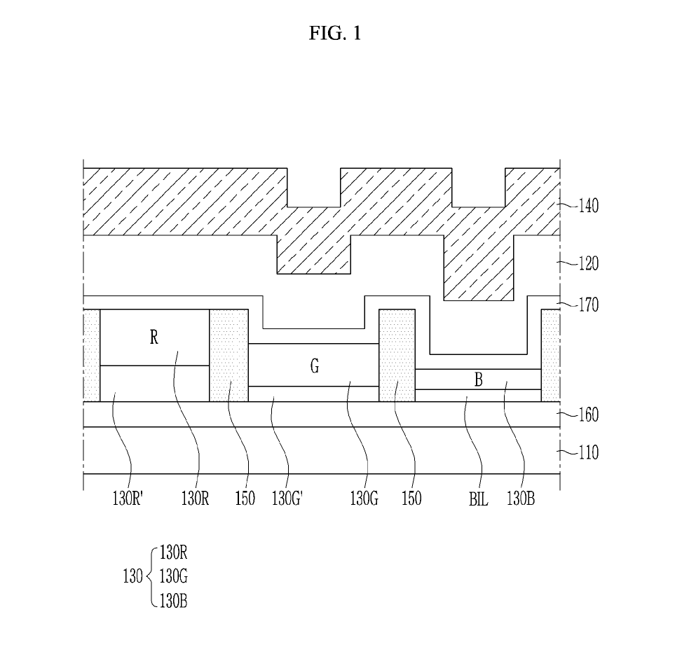

FIG. 1 is a view schematically showing a structure of an organic light emitting diode according to the present exemplary embodiment. As shown in FIG. 1, an organic light emitting diode according to the present exemplary embodiment includes a first electrode 110, a second electrode 120, an organic emission layer 130, and a capping layer 140.

The first electrode 110 is formed on the substrate and may serve an anode function to emit electrons into the organic emission layer 130. However, it is not limited thereto, and when the second electrode 120 functions as the anode, the first electrode 110 may be a cathode.

The organic light emitting diode according to the present exemplary embodiment may be a top emission organic light emitting diode. Accordingly, the first electrode 110 may serve as a reflection layer not emitting light emitted from the organic emission layer 130 to a rear surface. Here, the reflection layer means a layer having a characteristic of reflecting the light emitted from the organic emission layer 130 so as to be emitted through the second electrode 120 to the outside. The reflection characteristic may mean that reflectivity of incident light is about 70% or more to about 100% or less, or about 80% or more to about 100% or less.

The first electrode 110 according to the present exemplary embodiment may include silver (Ag), aluminum (Al), chromium (Cr), molybdenum (Mo), tungsten (W), titanium (Ti), gold (Au), palladium (Pd), or alloys thereof to be used as the reflection layer while having the anode function, and may be a triple layer structure of silver (Ag)/indium tin oxide (ITO)/silver (Ag) or indium tin oxide (ITO)/silver (Ag)/indium tin oxide (ITO).

The second electrode 120 is disposed to overlap the first electrode 110 via the organic emission layer 130 interposed therebetween with the first electrode 110, as described later. The second electrode 120 according to the present exemplary embodiment may function as the cathode. However, it is not limited thereto, and when the first electrode 110 functions as the cathode, the second electrode 120 may be the anode.

The second electrode 120 according to the present exemplary embodiment may be a transflective electrode for the light emitted from the organic emission layer 130 to be emitted to the outside. Here, the transflective electrode means an electrode having a transflective characteristic transmitting part of the light incident to the second electrode 120 and reflecting a remaining part of the light to the first electrode 110. Here, the transflective characteristic may mean that the reflectivity for the incident light is about 0.1% or more to about 70% or less, or about 30% or more to about 50% or less.

The second electrode 120 according to the present exemplary embodiment may include an oxide such as ITO or IZO, or silver (Ag), magnesium (Mg), aluminum (Al), chromium (Cr), molybdenum (Mo), tungsten (W), titanium (Ti), gold (Au), palladium (Pd), or alloys to have the transflective characteristic and simultaneously to have electrical conductivity.

In this case, the second electrode 120 of the present exemplary embodiment to smoothly emit the light emitted from the organic emission layer 130 to the outside, particularly, to smoothly emit the light of a blue color range, may have light transmittance of about 20% or more for light of a 430 nm to 500 nm wavelength. This is a minimum light transmittance to realize a color through the organic light emitting diode according to the present exemplary embodiment, and closer to 100% is preferred.

In the organic emission layer 130, holes and electrons respectively transmitted from the first electrode 110 and the second electrode 120 meet, thereby forming an exciton to emit light. In FIG. 1, the organic emission layer 130 includes a blue emission layer 130B, and may further include a red emission layer 130R and a green emission layer 130G, or may have a single layer structure in which the blue emission layer 130B, the red emission layer 130R, and the green emission layer 130G are respectively disposed in the same layer on the first electrode 110.

Blue, red, and green are three primary colors to realize the color, and combinations thereof may realize various colors. The blue emission layer 130B, the red emission layer 130R, and the green emission layer 130G respectively form a blue pixel, a red pixel, and a green pixel. The blue emission layer 130B, the red emission layer 130R, and the green emission layer 130G may be disposed on an upper surface of the first electrode 110.

A hole transmission layer 160 may be further included between the first electrode 110 and the organic emission layer 130. The hole transmission layer 160 may include at least one of a hole injection layer and a hole transport layer. The hole injection layer facilitates the injection of the hole from the first electrode 110, and the hole transport layer transports the hole from the hole injection layer. The hole transmission layer 160 may be formed of a dual layer in which the hole transport layer is formed on the hole injection layer, and may be formed of the single layer in which the material forming the hole injection layer and the material forming the hole transport layer are mixed.

An electron transmission layer 170 may be further included between the second electrode 120 and the organic emission layer 130. The electron transmission layer 170 may include at least one of an electron injection layer and an electron transport layer. The electron injection layer facilitates the injection of the electron from the second electrode 120, and the electron transport layer transports the electron transmitted from the electron injection layer. The electron transmission layer 170 may be formed of a dual layer in which the electron transport layer is formed on the electron injection layer, and may be formed of the single layer in which the material forming the electron injection layer and the material forming the electron transport layer are mixed.

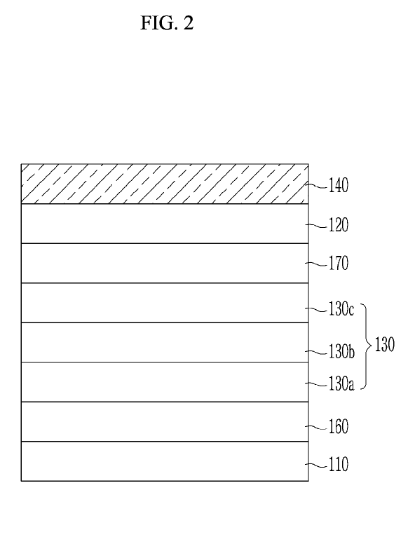

However, the inventive concept is not limited thereto, and the organic light emitting diode according to the exemplary variation may include the organic emission layer 130 having the multi-layered structure. This will be further described with reference to FIG. 2.

FIG. 2 schematically shows the organic light emitting diode including the organic emission layer 130 having the multi-layered structure according to another exemplary embodiment of the described technology.

In the exemplary embodiment shown in FIG. 2, configurations except for the organic emission layer 130 are similar to the configurations of the organic light emitting diode according to the exemplary embodiment described with reference to FIG. 1. Accordingly, the first electrode 110 and the second electrode 120 are disposed to be overlapped, the organic emission layer 130 is between the first electrode 110 and the second electrode 120, the electron transmission layer 170 is disposed between the organic emission layer 130 and the second electrode 120, and the capping layer 140 is on the second electrode 120.

In this case, the organic emission layer 130 according to the present exemplary embodiment is formed by depositing a plurality of layers 130a, 130b, and 130c. The layers 130a, 130b, and 130c of the organic emission layer 130 respectively represent the different colors, thereby emitting white-colored light by combination.

As shown in FIG. 2, the organic emission layer 130 according to the present exemplary embodiment may have the three-layered structure in which three layers 130a, 130b, and 130c are deposited; however, the inventive concept is not limited thereto, and the organic emission layer 130 may have a structure made of two layers.

As one example, the organic emission layer 130 of the three-layered structure may include a blue emission layer 130a, a yellow emission layer 130b, and a blue emission layer 130c. However, this is not a limitation of the disclosed concept thereto, and any emission layer capable of emitting white light by the color combination may be included in an exemplary embodiment range of the described technology.

Also, although not shown in the drawing, in the case of the organic emission layer of a two-layered structure, each layer may include the blue emission layer and the yellow emission layer.

In addition, although not shown in the drawing, a charge generation layer may be between adjacent layers among the plurality of layers 130a, 130b, and 130c of FIG. 2.

In the display device using the organic light emitting diode according to the present exemplary embodiment, to convert the emitted white light into the other colors, a color filter layer disposed on the second electrode 120 may be further included.

For example, the color filter layer may convert the white light passing through the second electrode 120 into blue, red, or green, and for this, a plurality of sub-color filter layers respectively corresponding to a plurality of sub-pixels of the organic light emitting diode may be included. The color filter layer converts the color of the light emitted from the second electrode 120 such that various position designs may be possible if the color filter layer is only disposed on the second electrode 120.

To protect the display device from external moisture or oxygen, the color filter layer may be disposed on or under an encapsulation layer, and various arrangement structures of the color filter layers are possible, such that the embodiment range of the present exemplary embodiment may be applied to these arrangement structures.

The organic light emitting diode according to the exemplary embodiment shown in FIG. 2 is the same as the exemplary embodiment shown in FIG. 1 except for the emission of white light by including the organic emission layer 130 made of the plurality of layers 130a, 130b, and 130c stacked on top of one another. Therefore, the following is described with reference to the organic light emitting diode shown in FIG. 1. The following description for the organic light emitting diode may be equally applied to the exemplary embodiment shown in FIG. 2.

The blue emission material included in the blue emission layer 130B according to the present exemplary embodiment has a range of a peak wavelength of about 430 nm to 500 nm in a photoluminescence (PL) spectrum.

As shown in FIG. 1, an auxiliary layer BIL to increase efficiency of the blue emission layer 130B may be under the blue emission layer 130B. The auxiliary layer BIL may have the function of increasing the efficiency of the blue emission layer 130B by controlling a hole charge balance.

Similarly, as shown in FIG. 1, a red resonance auxiliary layer 130R′ and a green resonance auxiliary layer 130G′ may be respectively under the red emission layer 130R and the green emission layer 130G. The red resonance auxiliary layer 130R′ and the green resonance auxiliary layer 130G′ are added in order to match a resonance distance for each color. Alternatively, the separate resonance auxiliary layer may not be formed under the blue emission layer 130B and the auxiliary layer BIL.

A pixel definition layer 150 may be on the first electrode 110. The pixel definition layer 150, as shown in FIG. 1, is respectively between the blue emission layer 130B, the red emission layer 130R, and the green emission layer 130G, thereby dividing the emission layers for each color.

The capping layer 140 is formed on the second electrode 120 to control a length of a light path of the element, thereby adjusting an optical interference distance. In this case, the capping layer 140 according to the present exemplary embodiment, differently from the auxiliary layer BIL, the red resonance auxiliary layer 130R′, and the green resonance auxiliary layer 130G′, as shown in FIG. 1, may be commonly provided in each of the blue pixel, the red pixel, and the green pixel.

The organic emission layer 130 according to the present exemplary embodiment, particularly, in reaction to being exposed to light such as sunlight, is damaged by the wavelength near 405 nm such that the performance of the organic light emitting diode may be deteriorated. Accordingly, 405 nm is the wavelength of light that causes an organic light emitting diode to deteriorate, and will herein be referred to as “harmful wavelength.” The capping layer 140 according to the present exemplary embodiment is formed by including a material that blocks the light near the 405 nm that is the harmful wavelength region among the light incident to the organic emission layer 130 to prevent the degradation of the organic emission layer 130 included in the organic light emitting diode.

To block the light in the 405 nm region as the harmful wavelength region, the capping layer 140 according to the present exemplary embodiment may have k₁ of 0.25 or more as an absorption rate at 405 nm. When k₁ is less than 0.25, the capping layer 140 according to the present exemplary embodiment does not effectively block the light of a 405 nm wavelength of the harmful wavelength region such that it is difficult to obtain the effect of preventing the degradation of the organic emission layer 130.

According to the present exemplary embodiment, absorption rates k₁ and k₂ and a refractive index described below are values that are measured by using FILMETRICS F10-RT-UV equipment after forming the capping layer 140 according to the present exemplary embodiment by depositing the organic material on a silicon substrate as a thin film having a thickness of 70 nm.

As k₁ increases, more of the light in a 405 nm of the harmful wavelength region is blocked. As one example of the present exemplary embodiment, the material forming the capping layer 140 may be selected such that k₁ is 0.8 or less, and preferably, the material forming the capping layer 140 may be selected in a range such that k₁ is 1.0 or less. However, this is only one example, and the selection range of the material forming the capping layer 140 may be determined by considering various factors such as the thickness of the capping layer 140 and a usage environment.

On the other hand, the organic emission layer 130 according to the present exemplary embodiment has high transmittance for light of a 430 nm wavelength as the blue light while blocking the light of a 405 nm wavelength as being in the harmful wavelength region. Hence, the damaging wavelength is blocked without compromising the efficiency of the blue series light. For this, the capping layer 140 according to the present exemplary embodiment may have an absorption rate k₂ of less than 0.25 for the light of the 430 nm wavelength as the wavelength of the blue series light.

When k₂ is larger than 0.25, the ratio of the blue light that is absorbed by the capping layer 140 is increased such that it may be difficult to achieve the various colors through the organic light emitting diode according to the present exemplary embodiment.

As k₂ gets closer to 0, the ratio of the blue light absorbed by the capping layer 140 is decreased such that the efficiency of the blue light may be increased.

In this case, the capping layer 140 according to the present exemplary embodiment may include a material having the high refractive index for the blue series light. This way, the emission efficiency in the blue region is not compromised. In detail, the capping layer 140 according to the present exemplary embodiment may have the refractive index of 2.0 or more in the wavelength range of 430 nm to 470 nm. If the refractive index of the capping layer 140 is increased, a resonance effect may be further generated by the refraction such that the emission efficiency may be increased.

To smoothly generate the resonance effect, the capping layer 140 according to the present exemplary embodiment may have a 200 nm or less (0 is not included) thickness. As one example, the capping layer 140 having a thickness of 60 nm to 80 nm may be formed, but this inventive concept is not limited thereto.

The capping layer 140 according to the present exemplary embodiment may include a material satisfying Equation A below. k₁−k₂>0.10

[Equation A]

In Equation A, k₁ is the absorption rate of the light having the wavelength of 405 nm, and k₂ is the absorption rate of the light having the wavelength of 430 nm.

In Equation A above, it is preferable that a difference between k₁ and k₂ is large. Accordingly, in Equation A, a difference between k₁ and k₂ may be larger than 0.1, which is a lower limit for the difference between the absorption rate k₁ for the light of a 405 nm wavelength as the harmful wavelength region and the absorption rate k₂ for the light of a 430 nm wavelength as the wavelength region of the blue series light.

In the case that the difference between k₁ and k₂ is smaller than 0.1, the light of the harmful wavelength region may still be blocked, but the emission efficiency of the blue light will likely decrease. Alternatively, the emission efficiency of the blue light may be maintained but the light of the harmful wavelength region may not be blocked effectively such that it is impossible to prevent the degradation of the organic emission layer 130.

Accordingly, to attain a desired level of emission efficiency of the blue light while effectively blocking the light in the harmful wavelength region, it is preferable that the difference between the absorption rate k₁ for the light of a 405 nm wavelength of the harmful wavelength region and the absorption rate k₂ for the light of a 430 nm wavelength of the wavelength region of the blue light is larger than 0.1. As the difference k₁−k₂ increases, the absorption rate of the light of the blue region decreases while a large percentage of the light of the harmful wavelength region gets absorbed, such that the overall efficiency may increase. It is further preferable that the difference between the absorption rate k₁ for the light of the 405 nm wavelength of the harmful wavelength region and the absorption rate k₂ for the light of the 430 nm wavelength of the wavelength region of the blue light is larger than 0.3, and more preferably, when the difference between the absorption rate k₁ for the light of the 405 nm wavelength of the harmful wavelength region and the absorption rate k₂ for the light of the 430 nm wavelength of the wavelength region of the blue light is larger than 0.5. The larger the difference between k₁ and k₂, the higher the light transmission of the blue region may be while more of the light of the harmful wavelength region is absorbed.

Therefore, it may be confirmed that larger than 0.1 for the difference between the absorption rate k₁ for the light of the 405 nm wavelength of the harmful wavelength region and the absorption rate k₂ for the light of the 430 nm wavelength of the wavelength region of the blue light is a threshold value of the lowest value capable of maintaining the efficiency transmission of the blue region light while absorbing the light of the harmful wavelength region.

The capping layer 140 according to the present exemplary embodiment as an organic material including a carbon atom and a hydrogen atom may include at least one selected from a group including an aromatic hydrocarbon compound including a substituent having at least one selected from a group including an oxygen atom, a sulfur atom, a nitrogen atom, a fluorine atom, a silicon atom, a chlorine atom, a bromine atom, and an iodine atom, an aromatic heterocyclic compound, and an amine compound.

A detailed example of a compound that may be used as the capping layer 140 according to the present exemplary embodiment may be a material according to Chemical Formula 1 to Chemical Formula 7 below.

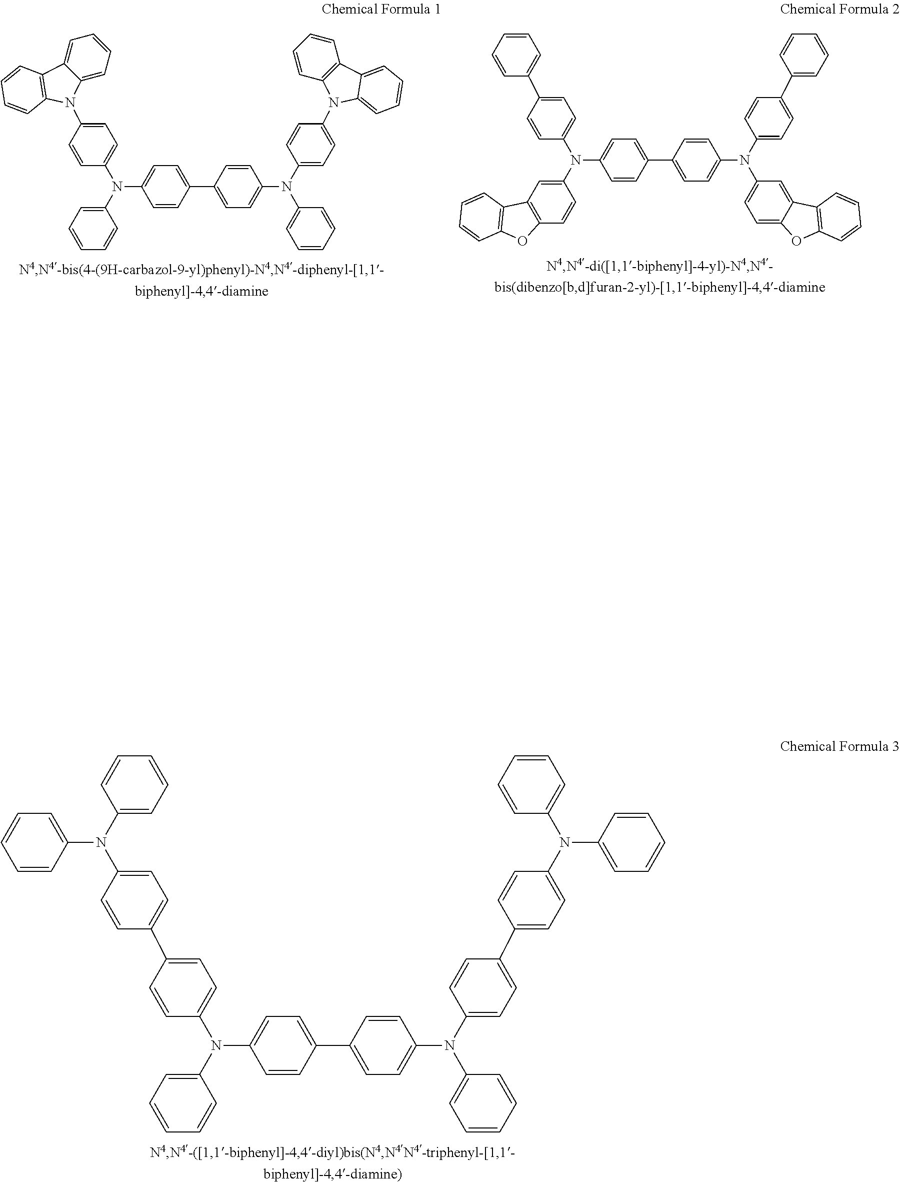 [SMILES: c1ccc(N(c2ccc(-c3ccc(N(c4ccccc4)c4ccc(-n5c6ccccc6c6ccccc65)cc4)cc3)cc2)c2ccc(-n3c4ccccc4c4ccccc43)cc2)cc1 ; c1ccc(-c2ccc(N(c3ccc(-c4ccc(N(c5ccc(-c6ccccc6)cc5)c5ccc6oc7ccccc7c6c5)cc4)cc3)c3ccc4oc5ccccc5c4c3)cc2)cc1 ; c1ccc(N(c2ccccc2)c2ccc(-c3ccc(N(c4ccccc4)c4ccc(-c5ccc(N(c6ccccc6)c6ccc(-c7ccc(N(c8ccccc8)c8ccccc8)cc7)cc6)cc5)cc4)cc3)cc2)cc1]

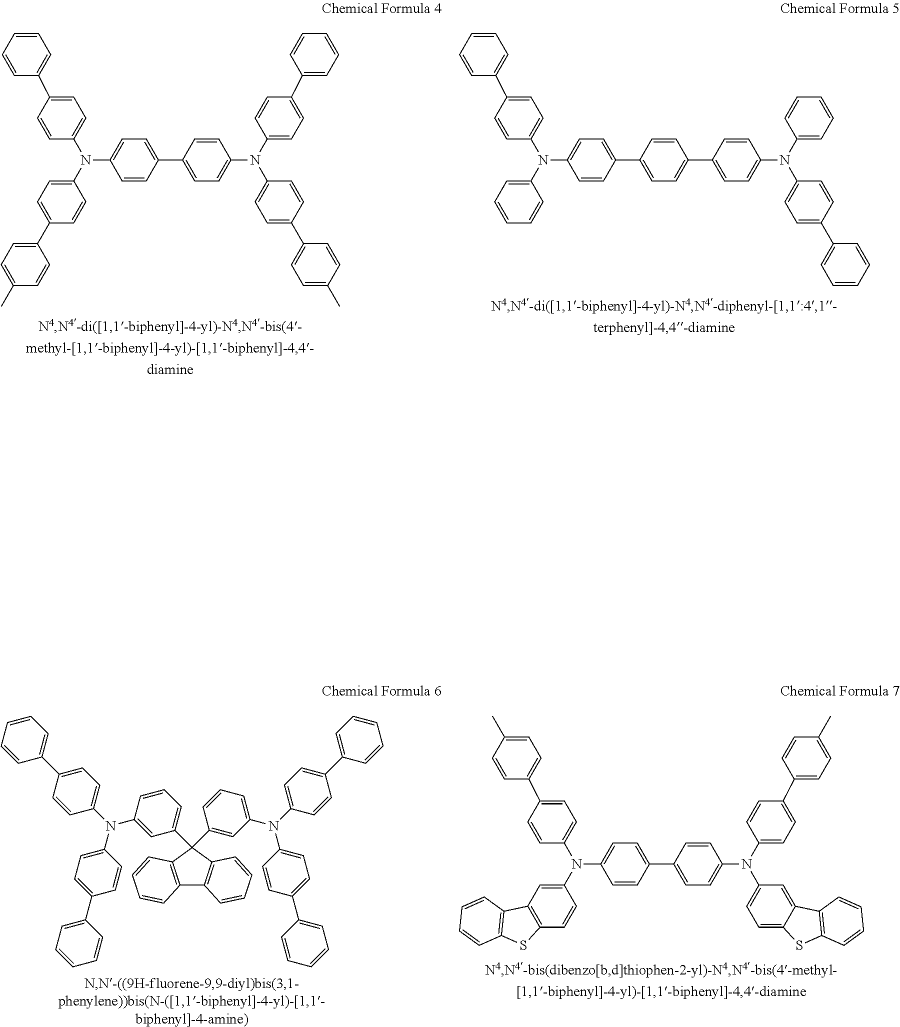 [SMILES: Cc1ccc(-c2ccc(N(c3ccc(-c4ccccc4)cc3)c3ccc(-c4ccc(N(c5ccc(-c6ccccc6)cc5)c5ccc(-c6ccc(C)cc6)cc5)cc4)cc3)cc2)cc1 ; c1ccc(-c2ccc(N(c3ccccc3)c3ccc(-c4ccc(-c5ccc(N(c6ccccc6)c6ccc(-c7ccccc7)cc6)cc5)cc4)cc3)cc2)cc1 ; c1ccc(-c2ccc(N(c3ccc(-c4ccccc4)cc3)c3cccc(C4(c5cccc(N(c6ccc(-c7ccccc7)cc6)c6ccc(-c7ccccc7)cc6)c5)c5ccccc5-c5ccccc54)c3)cc2)cc1 ; Cc1ccc(-c2ccc(N(c3ccc(-c4ccc(N(c5ccc(-c6ccc(C)cc6)cc5)c5ccc6sc7ccccc7c6c5)cc4)cc3)c3ccc4sc5ccccc5c4c3)cc2)cc1]

Hereinafter, to confirm the effect of the organic light emitting diode according to the present exemplary embodiment, among Chemical Formula 1 to Chemical Formula 7, Chemical Formula 1 to Chemical Formula 6 are selected as Exemplary Embodiment 1 to Exemplary Embodiment 6, and the materials such as Chemical Formula 8 and Chemical Formula 9 are selected as Comparative Example 1 and Comparative Example 2 to measure the absorption rate, the refractive index, and the blocking rate, and to confirm the blocking effect.

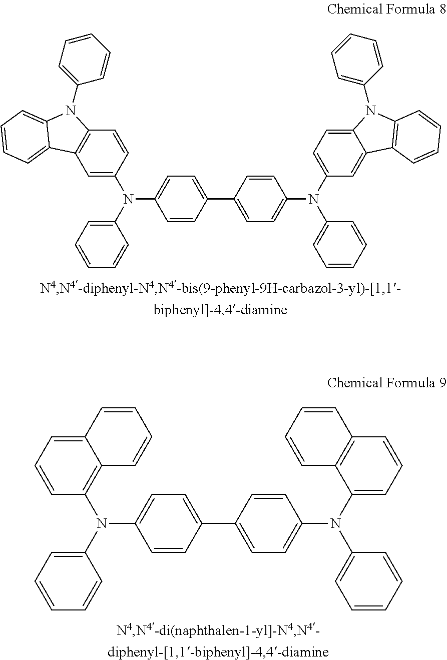 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; c1ccc(N(c2ccc(-c3ccc(N(c4ccccc4)c4cccc5ccccc45)cc3)cc2)c2cccc3ccccc23)cc1]

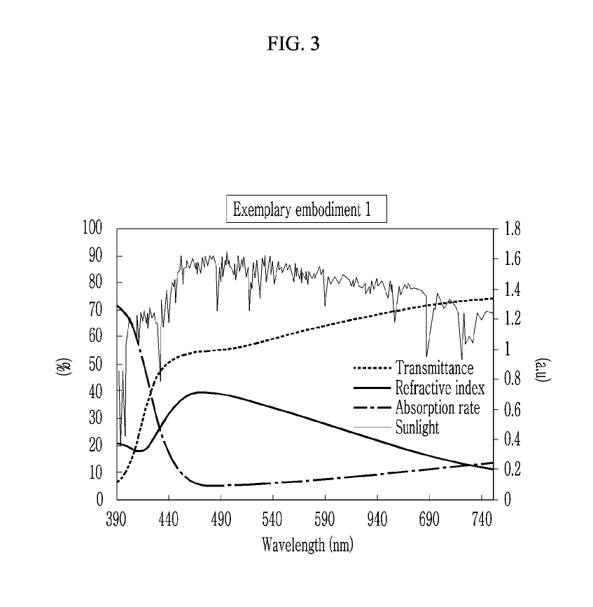

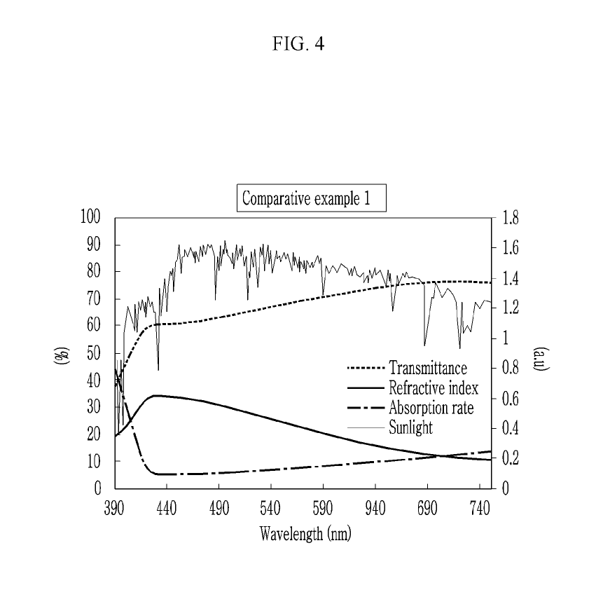

FIG. 3 is a graph showing an absorption rate, a refractive index, transmittance, and a sunlight spectrum of a capping layer material corresponding to Exemplary Embodiment 1, and FIG. 4 is a graph showing an absorption rate, a refractive index, transmittance, and a sunlight spectrum of a capping layer material corresponding to Comparative Example 1, while the absorption rate, the refractive index, and the blocking rate for each material corresponding to Exemplary Embodiment 1 to Exemplary Embodiment 6, and Comparative Example 1 and Comparative Example 2, are measured and the calculated results are summarized in Table 1. “Blocking rate” means that “(incident light−transmitted light)/incident light×100%.”

TABLE 1

| | | | | k₁ 405 | Blocking | Blocking |
|---|---|---|---|---|---|---|
| | k₁ | k₂ | n | nm-k₂ | rate | effect |
| Experiment | 405 nm | 430 nm | 450 nm | 430 nm | 405 nm | 405 nm |
| Exemplary | 0.539 | 0.134 | 2.248 | 0.405 | 83.80% | 1.66 |
| Embodiment | | | | | | |
| 1 | | | | | | |
| Exemplary | 0.511 | 0.089 | 2.177 | 0.422 | 82.49% | 1.64 |
| Embodiment | | | | | | |
| 2 | | | | | | |
| Exemplary | 0.459 | 0.067 | 2.254 | 0.392 | 79.00% | 1.57 |
| Embodiment | | | | | | |
| 3 | | | | | | |
| Exemplary | 0.730 | 0.216 | 2.269 | 0.514 | 91.52% | 1.82 |
| Embodiment | | | | | | |
| 4 | | | | | | |
| Exemplary | 0.754 | 0.228 | 2.299 | 0.526 | 90.50% | 1.80 |
| Embodiment | | | | | | |
| 5 | | | | | | |
| Exemplary | 0.673 | 0.227 | 2.310 | 0.446 | 87.50% | 1.74 |
| Embodiment | | | | | | |
| 6 | | | | | | |
| Comparative | 0.0800 | 0.00 | 1.920 | 0.080 | 50.29% | 1.00 |
| Example 1 | | | | | | |
| Comparative | 0.248 | 0.00 | 2.269 | 0.248 | 66.38% | 1.28 |
| Example 2 | | | | | | |

As described in Table 1, the material for the capping layer 140 according to Comparative Example 1 and Comparative Example 2 has an absorption rate k₁ of less than 0.25 in a 405 nm wavelength. Comparative Example 1 having k₂ of 0 satisfies the condition of the present exemplary embodiment. However, the refractive index n in a 450 nm wavelength is less than 2 and the difference between k₁ and k₂ according to Equation A is smaller than 0.1 in Comparative Example 1. In Comparative Example 1, the conditions of the capping layer 140 according to the present exemplary embodiment except for k₂ are not all satisfied. In Comparative Example 2, k₂ is 0 and the difference between k₁ and k₂ according to Equation A is larger than 0.1, however the material k₁ has a absorption rate k₁ of 0.248, which is smaller than 0.25.

In this case, based on the blocking rate that Comparative Example 1 blocks the light of the 405 nm wavelength of the harmful wavelength region, the blocking rates of Exemplary Embodiment 1 to Exemplary Embodiment 6 and Comparative Example 2 are relatively calculated and are described as the blocking effect.

Even if the other conditions are all satisfied like Comparative Example 2 and the k₁ is only less than 0.25, it may be confirmed that the blocking effect of blocking the 405 nm wavelength of the harmful wavelength region is increased by 20% or more compared with Comparative Example 1.

However, as shown in Table 1, in the case of Exemplary Embodiment 1 to Exemplary Embodiment 6, it may be confirmed the effect of blocking the light of the 405 nm wavelength of the harmful wavelength region is exerted with a ratio of over 50% at a minimum compared with Comparative Example 1.

Also, when comparing Comparative Example 1 with Exemplary Embodiment 1 to Exemplary Embodiment 6, with reference to Exemplary Embodiment 3 in which the blocking effect is measured to be lowest is increased by 57% compared with Comparative Example 1, it may be confirmed that Exemplary Embodiment 3 has an increased blocking effect by more than half with respect to Comparative Example 1.

Next, while exposing the organic light emitting diode including the capping layer 140 according to Exemplary Embodiment 1 to Exemplary Embodiment 6, and Comparative Example 1 and Comparative Example 2, to the light source including a 405 nm wavelength of the harmful wavelength region for a predetermined time, a result of comparing the degree of degradation of the organic emission layer 130 included in the organic light emitting diode is described in Table 2. The light source used according to the present exemplary embodiment is an artificial sunlight source emitting artificial light that is similar to the sunlight spectrum.

TABLE 2

| | | | Color |
|---|---|---|---|
| | | | temperature |
| | Color temperature (1 cycle: 8 h | | change |
| | light source exposure time) | | amount |
| Experiment | 0 cycle (0 h) | 4 cycle 32 h | (ΔK) |
| Exemplary | 7207K | 7257K | 50 |
| Embodiment 1 | | | |
| Exemplary | 7312K | 7373K | 61 |
| Embodiment 2 | | | |
| Exemplary | 7189K | 7270K | 81 |
| Embodiment 3 | | | |
| Exemplary | 7283K | 7293K | 10 |
| Embodiment 4 | | | |
| Exemplary | 7190K | 7202K | 12 |
| Embodiment 5 | | | |
| Exemplary | 7260K | 7283K | 23 |
| Embodiment 6 | | | |
| Comparative | 7136K | 7703K | 567 |
| Example 1 | | | |
| Comparative | 7334K | 7746K | 412 |
| Example 2 | | | |

Each sample is manufactured to have a color temperature of 7200 K, as measured in a 0 cycle exposure time. Next, if each sample is exposed to the light source including a 405 nm wavelength of the harmful wavelength region for a predetermined time, the organic emission layer 130 included in each sample is damaged by the harmful wavelength such the color temperature is changed. Accordingly, it may be considered that the degradation of the organic emission layer 130 is largely generated when the color temperature change amount is large.

As shown in Table 2, in the case of Comparative Example 1 and Comparative Example 2, a temperature change is more than 400 K. When the color temperature change amount is 400 K or more, the white color change may be detected by the user or by the naked eye such that the sample is considered to be a defective panel. In contrast, in the case of Exemplary Embodiment 1 to Exemplary Embodiment 6, the change in color temperature is small, in the range of 10 K to 80 K. which is very different from 400 K at which the color temperature change amount can be detected by the naked eye.

Accordingly, compared with Comparative Example 1 and Comparative Example 2, the light of the 405 nm wavelength as the harmful wavelength region is blocked by the capping layer 140 included in Exemplary Embodiment 1 to Exemplary Embodiment 6. The presence of the capping layer 140 decreases the degradation of the organic emission layer 130.

In the above, the organic light emitting diode according to the present exemplary embodiment has been described. According to the described technology, the degradation of the organic emission layer 130 may be prevented by blocking the light of the harmful wavelength region, and the organic light emitting diode in which the blue emission efficiency is not deteriorated may be provided.

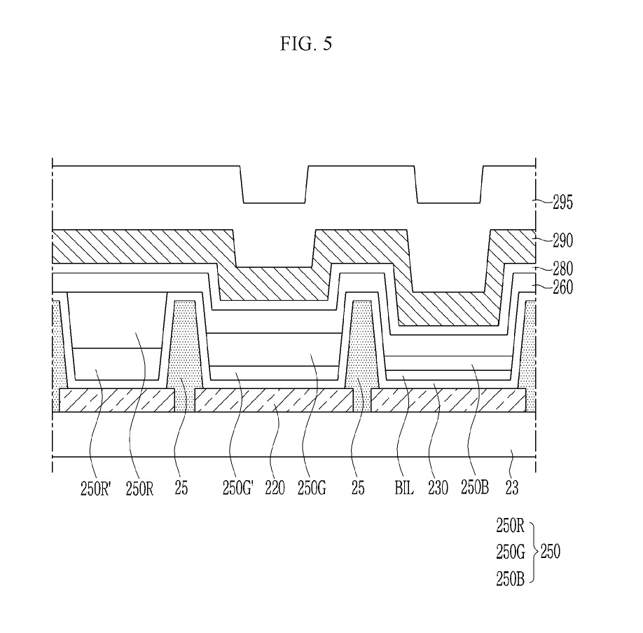

FIG. 5 is a cross-sectional view schematically showing a light emitting diode according to an exemplary embodiment of the described technology.

The exemplary embodiment to be described in FIG. 5 is almost the same as the exemplary embodiment described in FIG. 1. The differences will be explained first. Referring to FIG. 5, the light emitting diodes respectively corresponding to the red pixel, the green pixel, and the blue pixel are disposed on the substrate 23. The plurality of first electrodes 220 are disposed on the substrate 23 at the positions corresponding to each pixel, and the pixel definition layer 25 is formed between the adjacent first electrodes 220 among the plurality of first electrodes 220. The hole transmission layer 230 is formed on the first electrode 220 and the pixel definition layer 25. The red emission layer 250R, the green emission layer 250G, and the blue emission layer 250B may be formed of the organic emission layer or the inorganic material such as the quantum dot. In FIG. 5, it is shown that the red emission layer 250R, the green emission layer 250G, the blue emission layer 250B, the red resonance auxiliary layer 250R′, the green resonance auxiliary layer 250G′, and the auxiliary layer BIL are only disposed in the opening of the pixel definition layer 25, however at least part of each of the constituent elements may be formed on the pixel definition layer 25.

The electron transmission layer 170 described in the exemplary embodiment of FIG. 1 is embodied in the electron transport layer 260 and the electron injection layer 280 in the present exemplary embodiment. The electron transport layer 260 is disposed to be adjacent to the emission layer 250, and the electron injection layer 280 is disposed to be adjacent to the second electrode 290.

The electron transport layer 260 may include the organic material. For example, the electron transport layer 260 may be made of at least one selected from a group including Alq3 (tris(8-hydroxyquinolino)aluminum), PBD (2[4-biphenyl-5-[4-tert-butylphenyl]]-1,3,4-oxadiazole), TAZ (1,2,4-triazole), spiro-PBD (spiro-2[4-biphenyl-5-[4-tert-butylphenyl]]-1,3,4-oxadiazole), and BAlq(8-hydroxyquinoline beryllium salt), however it is not limited thereto.

The electron injection layer 280 may include lanthanum group elements. As the lanthanum group elements, ytterbium (Yb) having a work function of 2.6 eV, samarium (Sm) having the work function of 2.7 eV, or europium (Eu) having the work function of 2.5 eV may be used.

The contents described in the exemplary embodiment of FIG. 1 as well as the above-described contents may all be applied to the present exemplary embodiment. Also, the contents described in the exemplary embodiment of FIG. 2 may all be applied to the present exemplary embodiment.

However, the present exemplary embodiment corresponds to an exemplary embodiment describing the condition of the capping layer 295 required to prevent the emission layer 250 from being degraded in another aspect. To block the light of the 405 nanometer wavelength included in the harmful wavelength region, the capping layer 295 according to the present exemplary embodiment may satisfy Equation 1 below. The harmful wavelength region may be about 380 nanometers to 420 nanometers.

n*k(λ=405 nm)≧0.8 Equation 1

In Equation 1, n*k (λ=405 nm) represents the optical value that is a product of the refractive index in the 405 nanometer wavelength and the absorption coefficient. In the present disclosure, the absorption coefficient indicating the value k and the absorption rate are used with the same meaning.

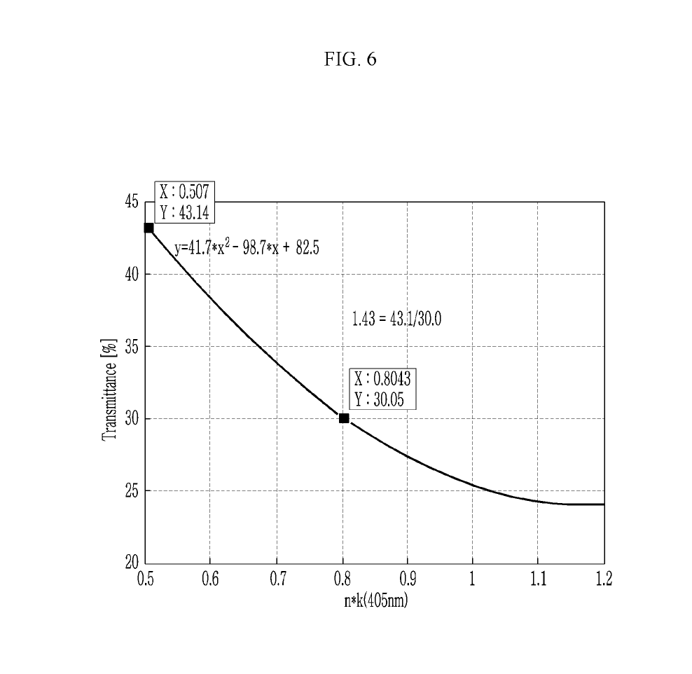

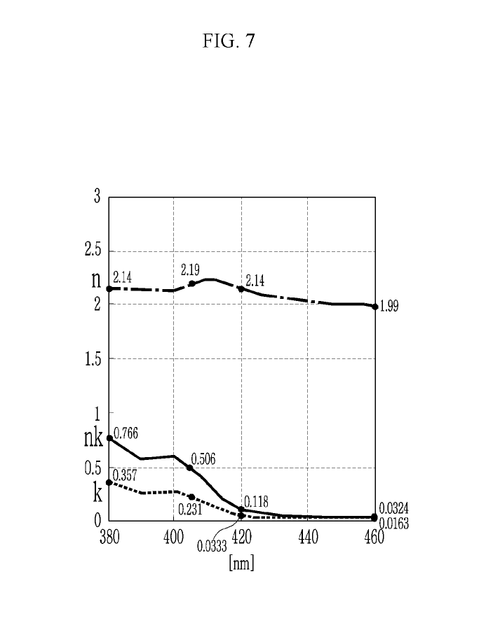

With regard to the number range represented in Equation 1, the mean for the number range will be described with reference to FIG. 6 and FIG. 7.

FIG. 6 is a graph showing a relation of an optical value (a product of a refractive index and an absorption coefficient) and a transmittance according to an exemplary embodiment of the described technology. FIG. 7 is a graph showing optical constants of a capping layer according to a comparative example.

Referring to FIG. 6, various materials having the different optical values (the product of the refractive index and the absorption coefficient) are exposed to a light source including the 405 nanometer wavelength to measure the transmittance, and a graph substantially satisfying a quadratic function shown in FIG. 6 may be obtained from the measured transmittance results.

Referring to FIG. 7, in a case of forming the capping layer 295 of FIG. 5 by using a compound represented by Chemical Formula 8 as a comparative example, the absorption coefficient k, the refractive index n, and the optical value (the product of the refractive index and the absorption coefficient) for the capping layer 295 depending on the wavelength of the light are shown. In the 405 nanometer wavelength included in the harmful wavelength region, the capping layer 295 according to the comparative example represents about 0.5 of the optical value (the product of the refractive index and the absorption coefficient). Again referring to FIG. 6, if the light emitting diode is formed by using the capping layer 295 according to the comparative example having about 0.5 of the optical value (the product of the refractive index and the absorption coefficient), transmittance of about 43% may be obtained.

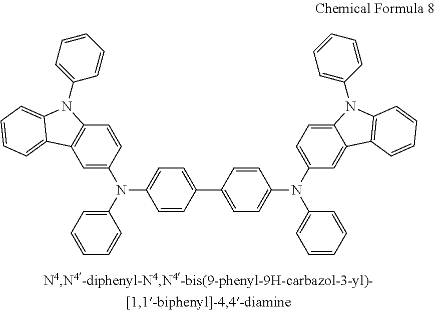 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1]

In contrast, to lower the transmittance of the light of the 405 nanometer wavelength to about 30% or less, it is preferable that the capping layer 295 according to the present exemplary embodiment has the optical value (the product of the refractive index and the absorption coefficient) of 0.8 or more. As the light transmittance of the 405 nanometer wavelength is lowered, the degradation degree of the emission layer may be lowered. When considering the correlation of the transmittance and the degradation degree of the emission layer, compared with the comparative example having the transmittance of about 43%, if the optical value of 0.8 or more may be obtained like the present exemplary embodiment having the transmittance of about 30%, it is possible to have the lifespan extension effect of about 1.43 times or more. The value X of 1.43 times is calculated through an inversely proportional relationship of 1:X=30:43 when the lifespan of the comparative example is 1.

To minimize the efficiency reduction for the blue light of the 460 nanometer wavelength while preventing the light of the 405 nanometer wavelength included in the harmful wavelength region, the capping layer 295 according to the present exemplary embodiment may satisfy Equation 2 below.

n*k(λ=460 nm)≦0.035 Equation 2

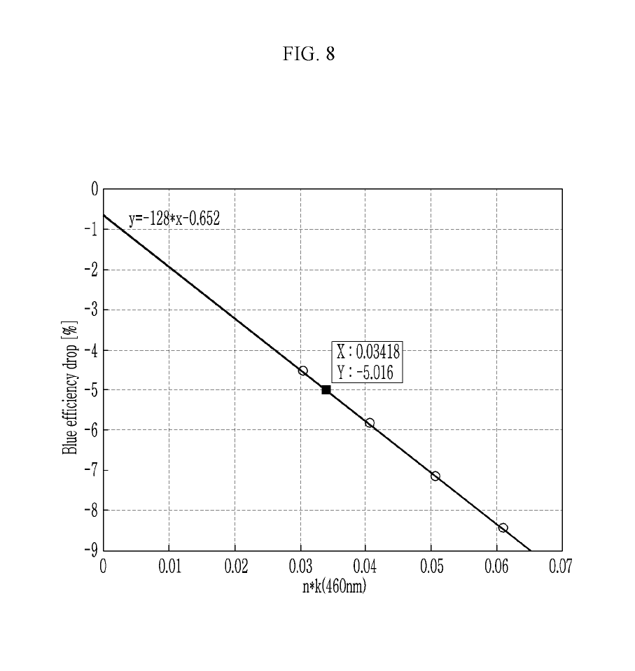

Related in the value range represented in Equation 2, the meaning of the value range will be described with reference to FIG. 8.

FIG. 8 is a graph showing a relation of an optical value (a product of a refractive index and an absorption coefficient) and a blue emission efficiency decreasing value according to an exemplary embodiment of the described technology. Referring to FIG. 8, as a comparative example, the capping layer 295 of FIG. 5 is formed of the compound represented by Chemical Formula 8. In this case, based on the light absorption rate of the 460 nanometer wavelength, a decreasing value of the light absorption rate in the 460 nanometer wavelength is measured for the various materials having the different optical values (the product of the refractive index and the absorption coefficient). By interpreting the measured light absorption rate decreasing value as the blue emission efficiency decreasing value, the graph substantially satisfying the straight line shown in FIG. 8 may be obtained.

Referring to FIG. 8, for the blue emission efficiency decreasing value to be about 5% or less compared with the comparative example, it is preferable that the capping layer according to the present exemplary embodiment has the optical value (the product of the refractive index and the absorption coefficient) of about 0.035 or less.

The capping layer according to the present exemplary embodiment may satisfy Equation 3 below.

n*k(λ=380 nm)≧2 Equation 3

In Equation 3, n*k (λ=380 nm) represents the optical value of the product of the refractive index and the absorption coefficient in the 380 nanometer wavelength.

By using the capping layer having the optical value of 2 or more in the 380 nanometer wavelength, the efficiency and the lifespan may be improved by blocking ultraviolet rays.

The capping layer satisfying the above-described Equation 1 and Equation 2 includes the first material, wherein the first material essentially includes a carbon atom and a hydrogen atom, and may include at least one selected from a group including an aromatic hydrocarbon compound containing a substituent having one or more selected from the group consisting of an oxygen atom, a sulfur atom, a nitrogen atom, a fluorine atom, a silicon atom, a chlorine atom, a bromine atom, and an iodine atom, an aromatic heterocyclic compound, and an amine compound.

The capping layer according to the present exemplary embodiment includes at least one among materials represented by Chemical Formula A and Chemical Formula B, while the optical value (the product of the refractive index and the absorption coefficient) satisfies at least one of Equation 1 and Equation 2.

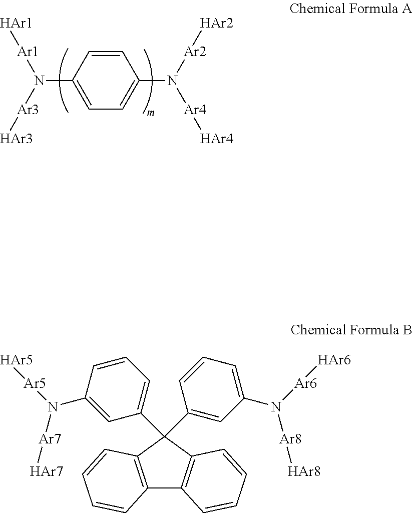

In Chemical Formula A, m is 2 to 4, in Chemical Formula A and Chemical Formula B, Ar1 to Ar8 are independently one of a single bond, phenylene, carbazole, dibenzothiophene, dibenzofuran, and biphenyl, and HAr1 to HAr8 are one of hydrogen, an alkyl group having 1 to 3 carbon atoms, a phenyl group, a carbazole group, a dibenzothiophene group, a dibenzofuran group, and a biphenyl group.

Chemical Formula A includes one among Chemical Formula A-1 to Chemical Formula A-3 below, and Chemical Formula B includes Chemical Formula B-1.

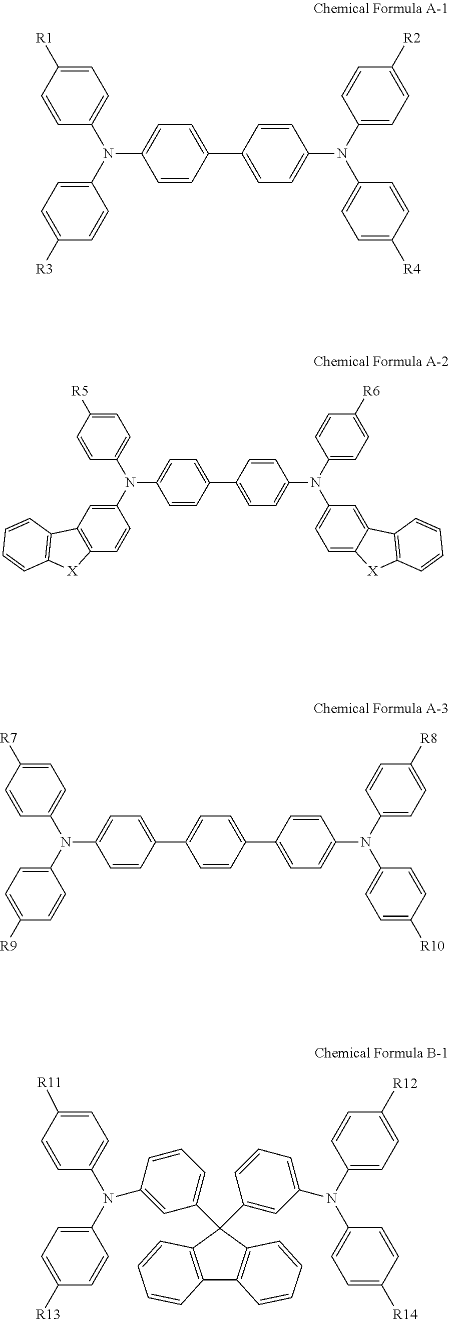

In Chemical Formula A-1 to Chemical Formula A-3, R1 to R10 are independently one of hydrogen, an alkyl group having 1 to 3 carbon atoms, a phenyl group, a carbazole group, a dibenzothiophene group, a dibenzofuran group, and a biphenyl group, and X is one of an oxygen atom, sulfur atom, and a nitrogen atom. In Chemical Formula B-1, R11 to R14 are independently one of hydrogen, an alkyl group having 1 to 3 carbon atoms, a phenyl group, a carbazole group, a dibenzothiophene group, a dibenzofuran group, and a biphenyl group.

In detail, the capping layer according to the present exemplary embodiment may include at least one among materials represented by Chemical Formula 1 to Chemical Formula 7 below.

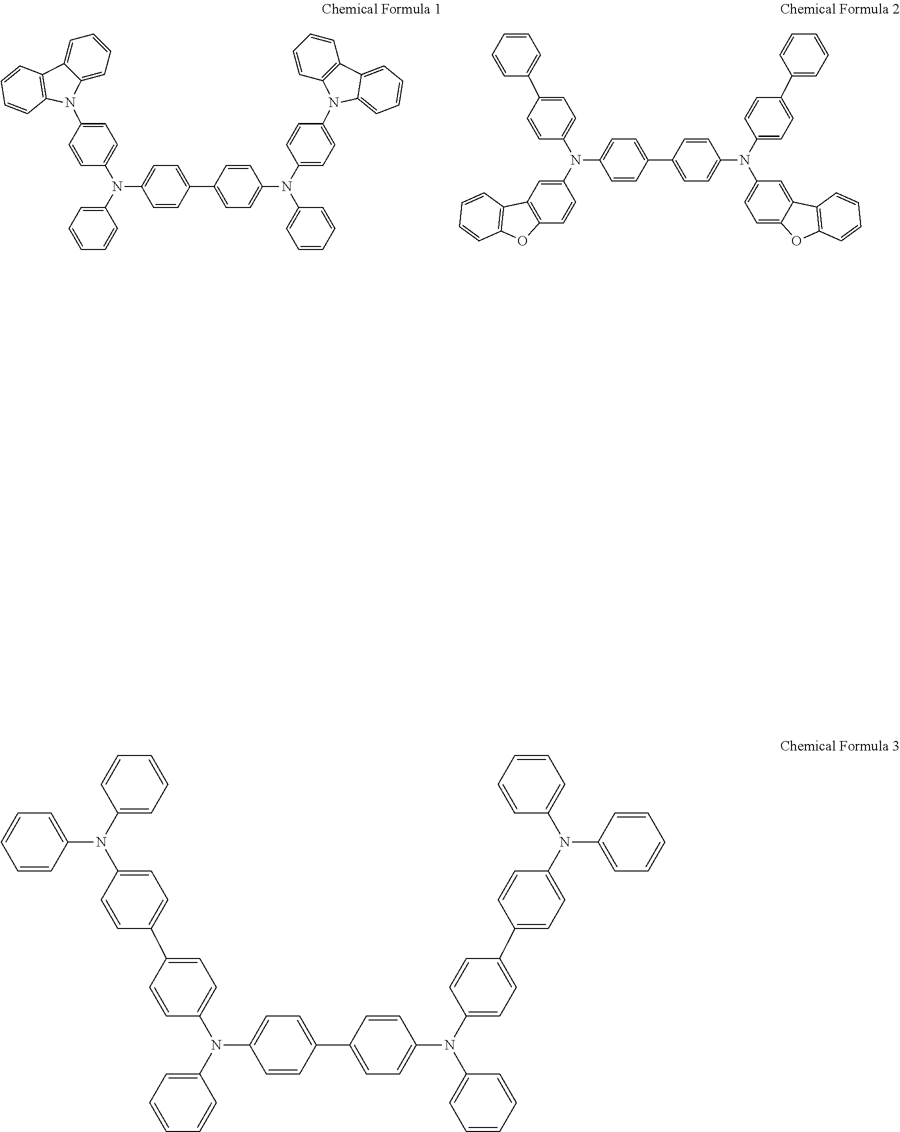 [SMILES: c1ccc(N(c2ccc(-c3ccc(N(c4ccccc4)c4ccc(-n5c6ccccc6c6ccccc65)cc4)cc3)cc2)c2ccc(-n3c4ccccc4c4ccccc43)cc2)cc1 ; c1ccc(-c2ccc(N(c3ccc(-c4ccc(N(c5ccc(-c6ccccc6)cc5)c5ccc6oc7ccccc7c6c5)cc4)cc3)c3ccc4oc5ccccc5c4c3)cc2)cc1 ; c1ccc(N(c2ccccc2)c2ccc(-c3ccc(N(c4ccccc4)c4ccc(-c5ccc(N(c6ccccc6)c6ccc(-c7ccc(N(c8ccccc8)c8ccccc8)cc7)cc6)cc5)cc4)cc3)cc2)cc1]

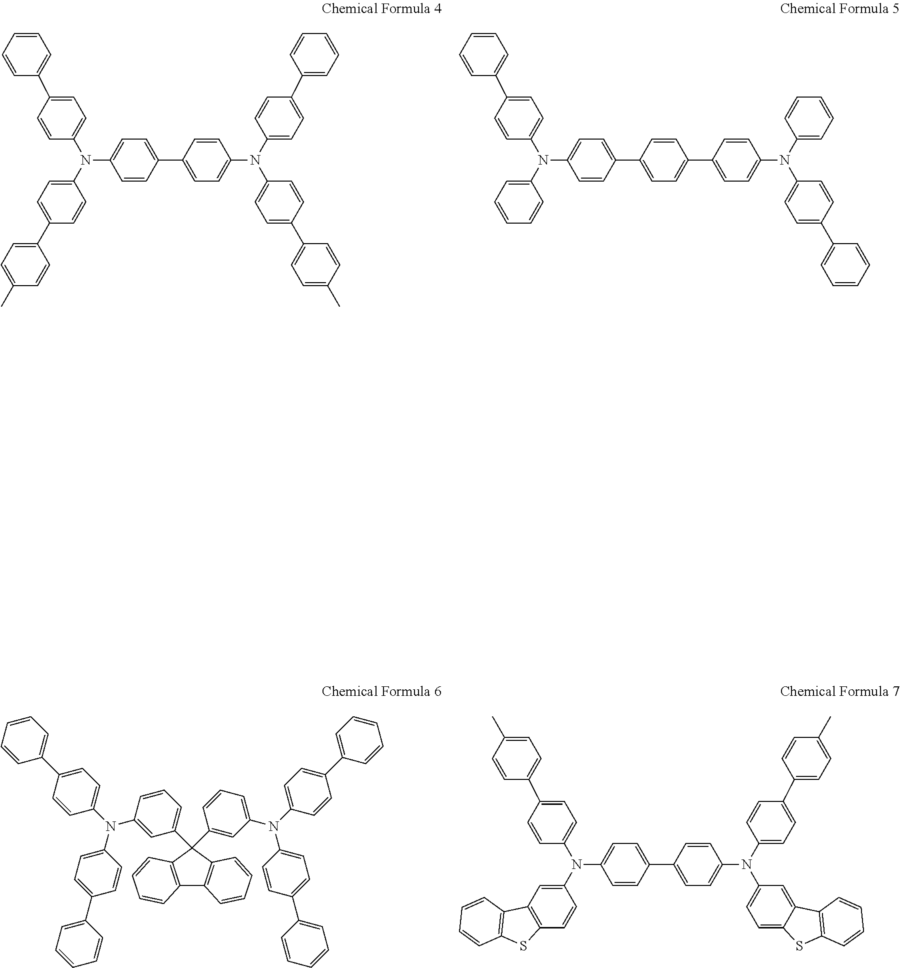 [SMILES: Cc1ccc(-c2ccc(N(c3ccc(-c4ccccc4)cc3)c3ccc(-c4ccc(N(c5ccc(-c6ccccc6)cc5)c5ccc(-c6ccc(C)cc6)cc5)cc4)cc3)cc2)cc1 ; c1ccc(-c2ccc(N(c3ccccc3)c3ccc(-c4ccc(-c5ccc(N(c6ccccc6)c6ccc(-c7ccccc7)cc6)cc5)cc4)cc3)cc2)cc1 ; c1ccc(-c2ccc(N(c3ccc(-c4ccccc4)cc3)c3cccc(C4(c5cccc(N(c6ccc(-c7ccccc7)cc6)c6ccc(-c7ccccc7)cc6)c5)c5ccccc5-c5ccccc54)c3)cc2)cc1 ; Cc1ccc(-c2ccc(N(c3ccc(-c4ccc(N(c5ccc(-c6ccc(C)cc6)cc5)c5ccc6sc7ccccc7c6c5)cc4)cc3)c3ccc4sc5ccccc5c4c3)cc2)cc1]

Additionally, the arranged materials forming the capping layer may satisfy Equation 3.

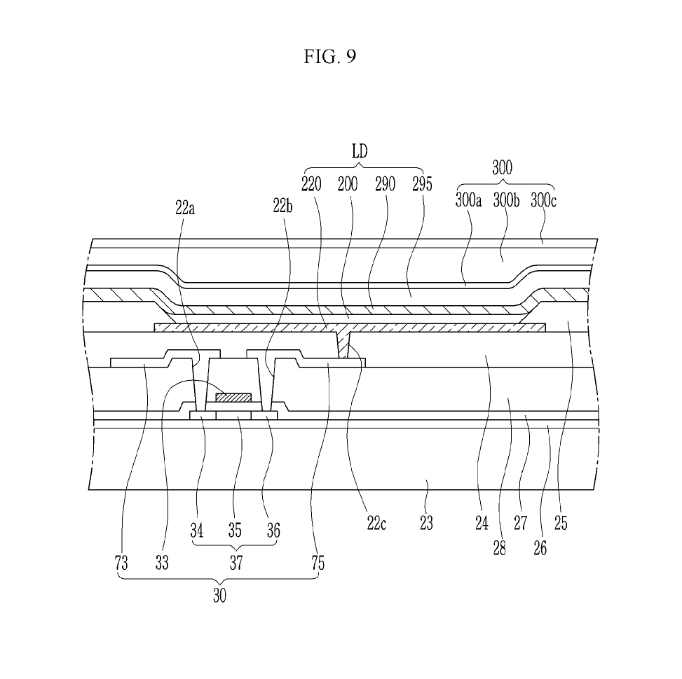

FIG. 9 is a cross-sectional view of a light emitting diode display according to another exemplary embodiment of the described technology.

Referring to FIG. 9, the display device according to the present exemplary embodiment includes a substrate 23, a driving transistor 30, a first electrode 220, a light emitting diode layer 200, and a second electrode 290. The first electrode 220 may be the anode and the second electrode 290 may be the cathode, however the first electrode 220 may be the cathode and the second electrode 290 may be the anode.

A substrate buffer layer 26 may be disposed on the substrate 23. The substrate buffer layer 26 serves to prevent penetration of impure elements and to planarize the surface, however, the substrate buffer layer 26 is not a necessary configuration, and may be omitted according to the type and process conditions of the substrate 23.

A driving semiconductor layer 37 is formed on the substrate buffer layer 26. The driving semiconductor layer 37 may be formed of a material including a polysilicon. Also, the driving semiconductor layer 37 includes a channel region 35 not doped with an impurity, and a source region 34 and a drain region 36 doped with an impurity at respective sides of the channel region 35. The doped ion materials may be P-type impurities such as boron (B), and B₂H₆ may be generally used. The impurities depend on the type of the thin film transistor.

A gate insulating layer 27 is disposed on the driving semiconductor layer 37. A gate wire including a driving gate electrode 33 is disposed on the gate insulating layer 27. The driving gate electrode 33 overlaps at least a portion of the driving semiconductor layer 37, and particularly, the channel region 35.

An interlayer insulating layer 28 covering the gate electrode 33 is formed on the gate insulating layer 27. A first contact hole 22a and a second contact hole 22b that respectively expose the source region 34 and the drain region 36 of the driving semiconductor layer 37 are formed in the gate insulating layer 27 and the interlayer insulating layer 28. A data wire including a driving source electrode 73 and a driving drain electrode 75 may be disposed on the interlayer insulating layer 28. The driving source electrode 73 and the driving drain electrode 75 are connected to the source region 34 and the drain region 36 of the driving semiconductor layer 37 through the first contact hole 22a and the second contact hole 22b formed in the interlayer insulating layer 28 and the gate insulating layer 27, respectively.

As described above, the driving thin film transistor 30 including the driving semiconductor layer 37, the driving gate electrode 33, the driving source electrode 73, and the driving drain electrode 75 is formed. The configuration of the driving thin film transistor 30 is not limited to the aforementioned example, and may be variously modified into other known configurations that may be easily implemented by those skilled in the art.

In addition, a planarization layer 24 covering the data wire is formed on the interlayer insulating layer 28. The planarization layer 24 serves to remove and planarize a step in order to increase emission efficiency of the light emitting diode to be formed thereon. The planarization layer 24 has a third contact hole 22c to electrically connect the driving drain electrode 75 and the first electrode that is described later.

This exemplary embodiment of the present disclosure is not limited to the above-described configuration, and one of the planarization layer 24 and the interlayer insulating layer 28 may be omitted in some cases.

The first electrode 220 of the light emitting diode LD is disposed on the planarization layer 24. The pixel definition layer 25 is disposed on the planarization layer 24 and the first electrode 220. The pixel definition layer 25 has an opening overlapping a part of the first electrode 220. In this case, the light emitting diode layer 100 may be disposed for each opening formed in the pixel definition layer 25.

On the other hand, the light emitting diode layer 200 is disposed on the first electrode 220. The light emitting diode layer 200 corresponds to the hole transmission layer 230, the emission layer 250, the electron transport layer 260, and the electron injection layer 280 in the light emitting diode described in FIG. 5.

In FIG. 9, the light emitting diode layer 200 is only disposed in the opening of the pixel definition layer 25, however as shown in FIG. 5, partial layers configuring the light emitting diode layer 200 may also be disposed on the upper surface of the pixel definition layer 25 like the second electrode 290.

A second electrode 290 and a capping layer 295 are disposed on the light emitting diode layer 200. The capping layer 295 may satisfy at least one of Equation 1 and Equation 2 described in FIG. 5 to FIG. 8, or may additionally satisfy Equation 3. The contents related to the above-described capping layer 295 may all be applied to the present exemplary embodiment.

A thin film encapsulation layer 300 is disposed on the capping layer 295. The thin film encapsulation layer 300 encapsulates the light emitting diode LD formed on the substrate 23 and a driving circuit to protect them from the outside.

The thin film encapsulation layer 300 includes a first inorganic layer 300a, an organic layer 300b, and a second inorganic layer 300c that are deposited one by one. In FIG. 9, the thin film encapsulation layer 300 is configured by alternately depositing two inorganic layers 300a and 300c and one organic layer 300b one by one. However, this is just an example and the inventive concept is not limited thereto. In a modified embodiment, the structure may include a plurality of the organic layer 300b and a plurality of the inorganic layer 300c. Although not shown, the light emitting diode display according to the present exemplary embodiment may further include a reflection blocking layer on the thin film encapsulation layer 300.

In Table 3 below, a comparative example represents the transmittance and the absorption rate in the 405 nanometer wavelength when forming the capping layer of the compound represented by Chemical Formula 8 with the 820 angstroms thickness and forming a SiNₓ layer with the 7000 angstroms thickness thereon. A Reference Example 1 is almost the same as the comparative example, but is a structure in which the capping layer thickness increases by 10%, a Reference Example 2 is a structure in which the thickness of the SiNx layer increases by 10%, and the Reference Examples 1 and 2 represent the transmittance and the absorption rate in the 405 nanometer wavelength for each of these structures. The Reference Example 3 is almost the same as the comparative example, but it is a deposition structure in which the capping layer thickness and the SiNₓ layer thickness increase by 10%, respectively. An Exemplary Embodiment 4 represents the transmittance and the absorption rate in the 405 nanometer wavelength in the structure only using a strong capping layer against sunlight. In the present disclosure, the strong capping layer means the capping layer formed by using the material satisfying at least one of above-described Equation 1 and Equation 2 or additionally satisfying Equation 3. An Exemplary Embodiment 2 represents the transmittance and the absorption rate in the 405 nanometer wavelength for a multi-layered structure in which the first capping layer is formed of the compound represented by Chemical Formula 8 with the 410 angstrom thickness, the second capping layer is formed of the strong capping layer with the 410 angstrom thickness, and the SiNₓ layer of the 7000 angstrom thickness is formed.

TABLE 3

| | | | | | | | Exemplary | Exemplary | | |
|---|---|---|---|---|---|---|---|---|---|---|
| | Comparative | | Reference | Reference | Reference | | Embodiment | Embodiment | | |
| | Example | | Example 1 | Example 2 | Example 3 | | 1 | 2 | | |
| Thickness | 820 | 7000 | 820*1.1 | 7000*1.1 | 820*1.1 | 7000*1.1 | 820 | 410 | 410 | 7000 |
| (Å) | | | | | | | | | | |

| | | | | | | |
|---|---|---|---|---|---|---|
| Trans- | 33.6 | 32.4 | 33 | 32.2 | 16.2 | 22.5 |
| mittance | 1 | −3.6% | −1.8% | −4.2% | −51.8% | −33.0% |
| 405 nm | | | | | | |
| Absorp- | 58.1 | 59.3 | 59.4 | 60.8 | 75.9 | 66 |
| tion rate | 1 | 2.1% | 2.2% | 4.6% | 30.6% | 13.6% |
| 405 nm | | | | | | |

In Table 3, even if the thicknesses of the capping layer and the SiNₓ layer according to the comparative example are changed, only an increase of the 2.1 to 2.2% degrees for the harmful wavelength absorption rate appears, however it may be confirmed that the absorption rate for the light of the 405 nanometer wavelength largely increases when forming the strong capping layer like the present exemplary embodiment. Also, in the multi-layered structure of the Exemplary Embodiment 2 including the strong capping layer, compared with the Exemplary Embodiment 1 only forming the strong capping layer, the increase degree of the light absorption rate of the 405 nanometer wavelength is not large, however it may be confirmed that the harmful wavelength absorption rate increases compared with the Reference Examples 1, 2, and 3 without the strong capping layer.

The substrate 23 of the light emitting diode display of the present exemplary embodiment may include a flexible material. Table 4 represents the transmittance of the light passing through each layer when irradiating the light of the 405 nanometer wavelength in each of a rigid light emitting diode display, the flexible light emitting diode display without the application of the strong capping layer and the flexible light emitting diode display including the strong capping layer.

TABLE 4

| | | Flexible | Flexible |
|---|---|---|---|
| | | light | light |
| | | emitting | emitting |
| | | diode | diode |
| | Rigid | display | display |
| | light | (without | (including |
| | emitting | strong | strong |
| 405 nanometer | diode | capping | capping |
| Light irradiation | display | layer) | layer) |
| Reflection preventing | 33.2% | 33.2% | 33.2% |
| layer transmission | | | |
| Encapsulation layer | 51.2% | 29.8% | 16.2% |
| and capping layer | | | |
| transmission | | | |
| Light emitting diode | 17.0% | 9.9% | 5.4% |
| arrival | | | |
| Excepted life span | 58% | 100% | 176% |

Referring to Table 4, in the flexible light emitting diode display including the strong capping layer, the light of the 405 nanometer wavelength included in the harmful wavelength reaching the light emitting diode is relatively very small. Accordingly, in the flexible light emitting diode display, if the strong capping layer is applied, there is an effect that the lifespan increase of 76% compared with the structure without the strong capping layer is produced.

While this invention has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims.

### DESCRIPTION OF SYMBOLS

110, 220: first electrode
120, 290: second electrode
130R, 250R: red emission layer
130G, 250G: green emission layer
130B, 250B: blue emission layer
BIL: auxiliary layer
140, 295: capping layer
25, 150: pixel definition layer

What is claimed is:

1. A light emitting diode comprising:
a first electrode;
a second electrode overlapping the first electrode;
an emission layer disposed between the first electrode and the second electrode; and
a capping layer disposed on the second electrode,
wherein the capping layer satisfies Equation 1 below:
n*k(λ=405 nm)≧0.8 Equation 1
wherein in Equation 1, n*k (λ=405 nm) represents an optical value that is a product of a refractive index and an absorption coefficient in a 405 nanometer wavelength.

2. The light emitting diode of claim 1, wherein
the capping layer satisfies Equation 2 below:
n*k(λ=460 nm)≦0.035 Equation 2
wherein in Equation 2, n*k (λ=460 nm) represents an optical value that is a product of a refractive index and an absorption coefficient in a 460 nanometer wavelength.

3. The light emitting diode of claim 2, wherein
the capping layer satisfies Equation 3 below:
n*k(λ=380 nm)≧2 Equation 3
wherein in Equation 3, n*k (λ=380 nm) represents an optical value that is a product of a refractive index and an absorption coefficient in a 380 nanometer wavelength.

4. The light emitting diode of claim 2, wherein
the capping layer comprises a first material,
the first material comprises a carbon atom and a hydrogen atom, and further includes
one or more selected from a group including an aromatic hydrocarbon compound including at least one substituent selected from a group including an oxygen atom, a sulfur atom, a nitrogen atom, a fluorine atom, a silicon atom, a chlorine atom, a bromine atom, and an iodine atom, an aromatic heterocyclic compound, and an amine compound, and
the optical value (a product of a refractive index and an absorption coefficient) of the first material satisfies at least one of Equation 1 and Equation 2.

5. The light emitting diode of claim 2, wherein
the capping layer comprises at least one among material represented by Chemical Formula A and Chemical Formula B while the optical value (the product of the refractive index and the absorption coefficient) of the capping layer satisfies at least one of Equation 1 and Equation 2: 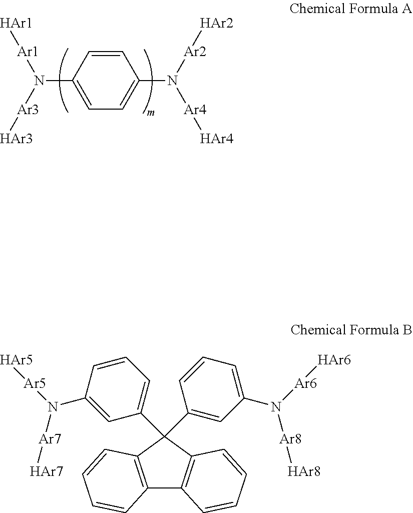
(in Chemical Formula A, m is 2 to 4,
in Chemical Formula A and Chemical Formula B,
Ar1 to Ar8 are independently one of a single bond, phenylene, carbazole, dibenzothiophene, dibenzofuran, and biphenyl,
HAr1 to HAr8 are one of hydrogen, an alkyl group having 1 to 3 carbon atoms, a phenyl group, a carbazole group, a dibenzothiophene group, a dibenzofuran group, and a biphenyl group).

6. The light emitting diode of claim 5, wherein
Chemical Formula A comprises one among Chemical Formula A-1 to Chemical Formula A-3, and Chemical Formula B comprises Chemical Formula B-1: 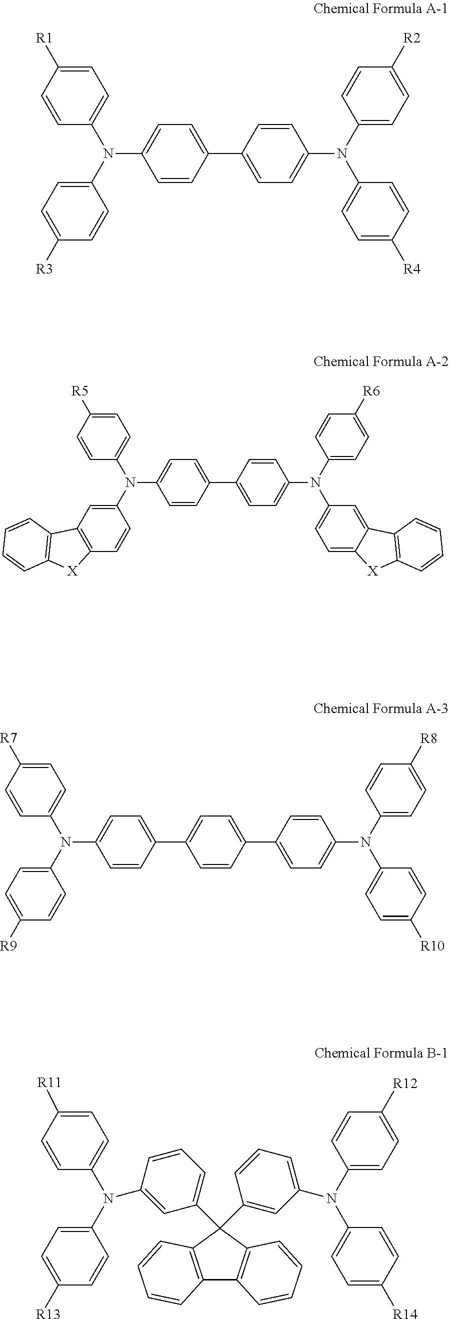
(in Chemical Formula A-1 to Chemical Formula A-3, R1 to R10 are independently one of hydrogen, an alkyl group having 1 to 3 carbon atoms, a phenyl group, a carbazole group, a dibenzothiophene group, a dibenzofuran group, and a biphenyl group, and X is one of an oxygen atom, a sulfur atom, and a nitrogen atom, and
in Chemical Formula B-1, R11 to R14 are independently one of hydrogen, an alkyl group having 1 to 3 carbon atoms, a phenyl group, a carbazole group, a dibenzothiophene group, a dibenzofuran group, and a biphenyl group).

7. The light emitting diode of claim 6, wherein
the capping layer includes at least one among materials represented by Chemical Formula 1 to Chemical Formula 7:

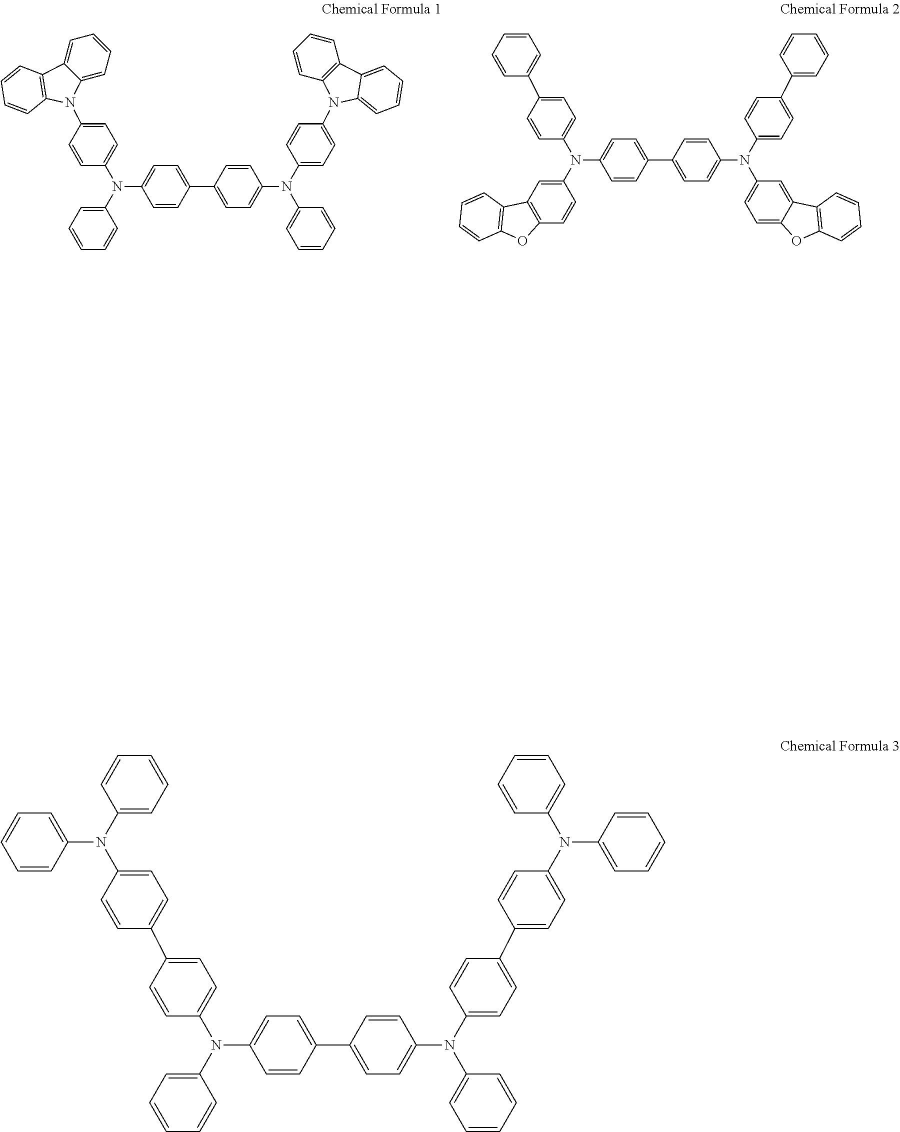 [SMILES: c1ccc(N(c2ccc(-c3ccc(N(c4ccccc4)c4ccc(-n5c6ccccc6c6ccccc65)cc4)cc3)cc2)c2ccc(-n3c4ccccc4c4ccccc43)cc2)cc1 ; c1ccc(-c2ccc(N(c3ccc(-c4ccc(N(c5ccc(-c6ccccc6)cc5)c5ccc6oc7ccccc7c6c5)cc4)cc3)c3ccc4oc5ccccc5c4c3)cc2)cc1 ; c1ccc(N(c2ccccc2)c2ccc(-c3ccc(N(c4ccccc4)c4ccc(-c5ccc(N(c6ccccc6)c6ccc(-c7ccc(N(c8ccccc8)c8ccccc8)cc7)cc6)cc5)cc4)cc3)cc2)cc1]

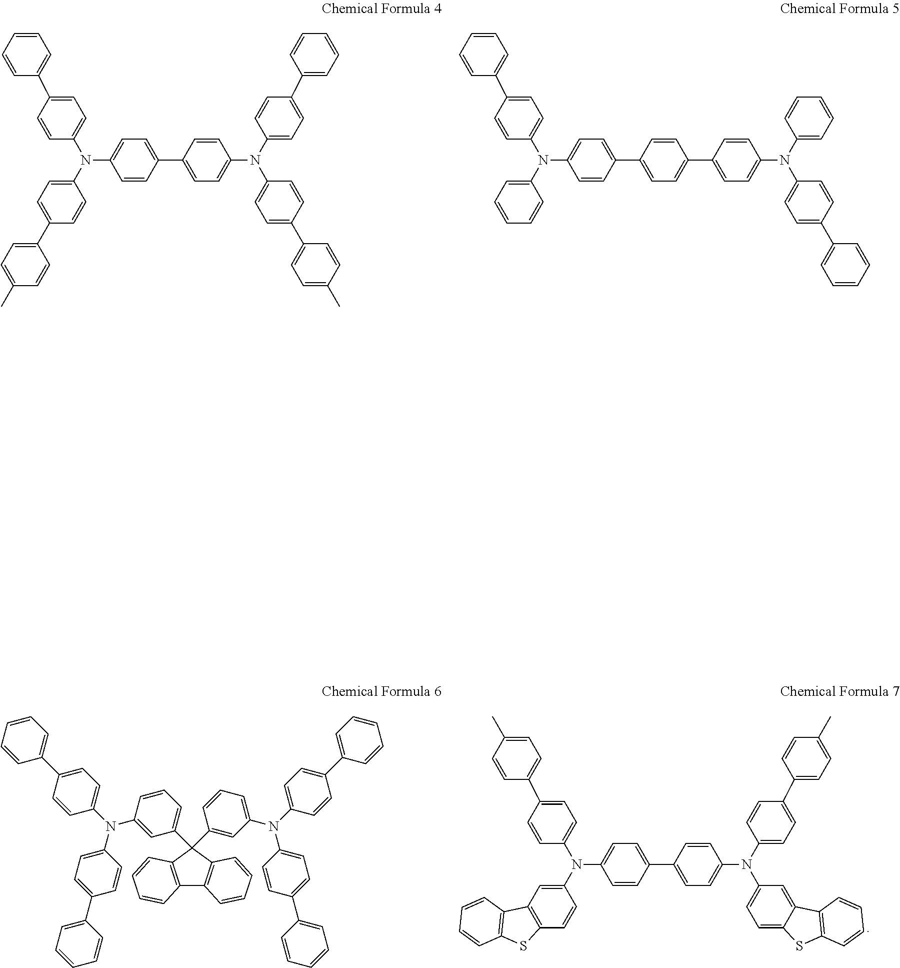 [SMILES: Cc1ccc(-c2ccc(N(c3ccc(-c4ccccc4)cc3)c3ccc(-c4ccc(N(c5ccc(-c6ccccc6)cc5)c5ccc(-c6ccc(C)cc6)cc5)cc4)cc3)cc2)cc1 ; c1ccc(-c2ccc(N(c3ccccc3)c3ccc(-c4ccc(-c5ccc(N(c6ccccc6)c6ccc(-c7ccccc7)cc6)cc5)cc4)cc3)cc2)cc1 ; c1ccc(-c2ccc(N(c3ccc(-c4ccccc4)cc3)c3cccc(C4(c5cccc(N(c6ccc(-c7ccccc7)cc6)c6ccc(-c7ccccc7)cc6)c5)c5ccccc5-c5ccccc54)c3)cc2)cc1 ; Cc1ccc(-c2ccc(N(c3ccc(-c4ccc(N(c5ccc(-c6ccc(C)cc6)cc5)c5ccc6sc7ccccc7c6c5)cc4)cc3)c3ccc4sc5ccccc5c4c3)cc2)cc1]

8. The light emitting diode of claim 1, wherein
the capping layer has light transmittance of 30% or less in the 405 nanometer wavelength.

9. The light emitting diode of claim 1, wherein
the emission layer comprises a blue emission layer, a red emission layer, and a green emission layer, and
the capping layer respectively overlaps the blue emission layer, the red emission layer, and the green emission layer.

10. The light emitting diode of claim 1, wherein
the emission layer emits a white light by a combination of a plurality of layers representing colors that are different from each other.

11. A light emitting diode display comprising:
a substrate;
a transistor disposed on the substrate;
a light emitting diode connected to the transistor; and
an encapsulation layer disposed on the light emitting diode,
wherein the light emitting diode comprises a first electrode, a second electrode overlapping the first electrode, an emission layer disposed between the first electrode and the second electrode, and a capping layer disposed on the second electrode, and the capping layer satisfies Equation 1 below:
n*k(λ=405 nm)≧0.8 Equation 1
wherein in Equation 1, n*k (λ=405 nm) represents an optical value that is a product of a refractive index and an absorption coefficient in a 405 nanometer wavelength.

12. The light emitting diode display of claim 11, wherein
the capping layer satisfies Equation 2 below:
n*k(λ=460 nm)≦0.035 Equation 2
wherein in Equation 2, n*k (λ=460 nm) represents an optical value that is a product of a refractive index and an absorption coefficient in a 460 nanometer wavelength.

13. The light emitting diode display of claim 12, wherein
the capping layer comprises a compound represented by one among Chemical Formula A-1 to Chemical Formula A-3, and Chemical Formula B-1, while the optical value (the product of the refractive index and the absorption coefficient) satisfies at least one of Equation 1 and Equation 2: 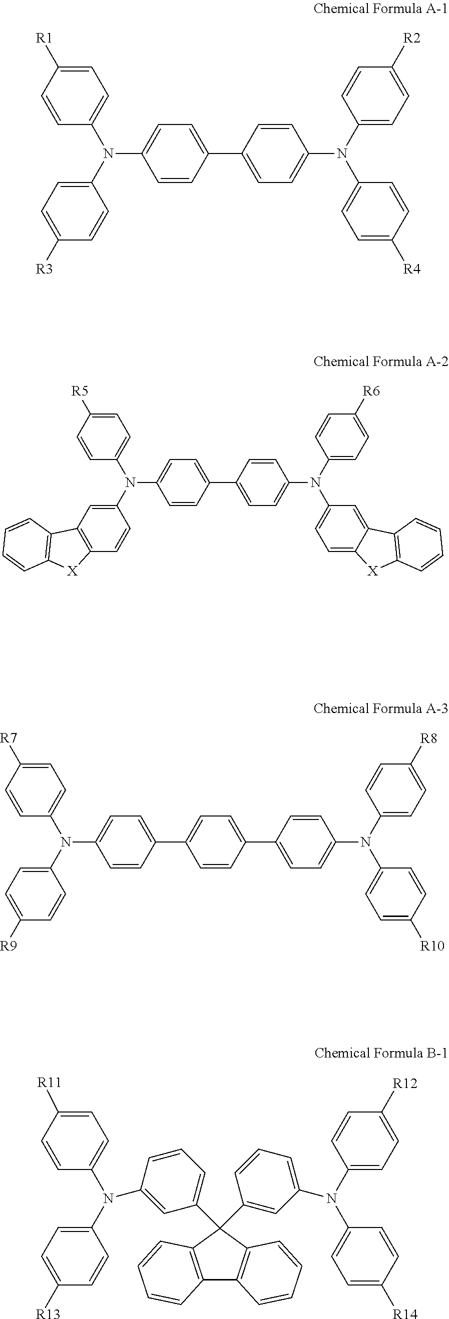
(in Chemical Formula A-1 to Chemical Formula A-3, R1 to R10 are independently one of hydrogen, an alkyl group having 1 to 3 carbon atoms, a phenyl group, a carbazole group, a dibenzothiophene group, a dibenzofuran group, and a biphenyl group, and X is one of an oxygen atom, a sulfur atom, and a nitrogen atom, and
in Chemical Formula B-1, R11 to R14 are independently one of hydrogen, an alkyl group having 1 to 3 carbon atoms, a phenyl group, a carbazole group, a dibenzothiophene group, a dibenzofuran group, and a biphenyl group).

14. The light emitting diode display of claim 12, wherein
the substrate comprises a flexible material.

15. The light emitting diode display of claim 14, wherein
the encapsulation layer comprises a structure in which an inorganic layer, an organic layer, and an inorganic layer are sequentially deposited.

16. An organic light emitting diode comprising:
a first electrode;
a second electrode overlapping the first electrode;
an organic emission layer disposed between the first electrode and the second electrode; and
a capping layer disposed on the second electrode,
wherein the capping layer has an absorption rate of 0.25 or more in a 405 nanometer wavelength,
the capping layer comprises at least one among materials represented by Chemical Formula A-1 to Chemical Formula A-3 and Chemical Formula B-1: 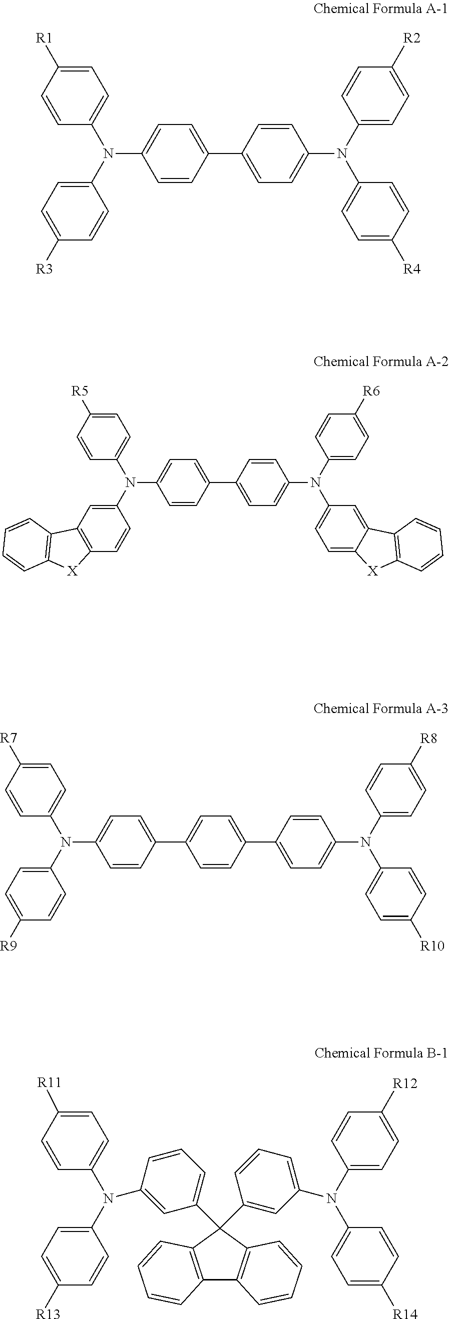
wherein in Chemical Formula A-1 to Chemical Formula A-3, R1 to R10 are independently one of hydrogen, an alkyl group having 1 to 3 carbon atoms, a phenyl group, a carbazole group, a dibenzothiophene group, a dibenzofuran group, and a biphenyl group, and X is one of an oxygen atom, a sulfur atom, and a nitrogen atom, and
in Chemical Formula B-1, R11 to R14 are independently one of hydrogen, an alkyl group having 1 to 3 carbon atoms, a phenyl group, a carbazole group, a dibenzothiophene group, a dibenzofuran group, and a biphenyl group.

17. The organic light emitting diode of claim 16, wherein
the capping layer has an absorption coefficient of 0.25 or less in a 430 nanometer wavelength.

18. The organic light emitting diode of claim 17, wherein
the capping layer satisfies Equation A below:
k₁−k₂>0.10 Equation A
in Equation A, k₁ is the absorption coefficient of the 405 nanometer wavelength, and k₂ is the absorption coefficient of the 430 nanometer wavelength.

19. The organic light emitting diode of claim 17, wherein
the capping layer has a refractive index of 2.0 or more in the wavelength range of about 430 nanometers to about 470 nanometers.

20. The organic light emitting diode of claim 16, wherein
the emission layer includes a blue emission layer, and
a light emission spectrum peak wavelength of a blue emission material included in the blue emission layer is about 430 nanometers to about 500 nanometers.

21. The organic light emitting diode of claim 16, wherein
the second electrode has a light transmittance of 20% or more in the wavelength range of about 430 nanometers to about 500 nanometers.

22. The organic light emitting diode of claim 16, wherein
the organic emission layer comprises a blue emission layer, a red emission layer, and a green emission layer, and
the capping layer respectively overlaps the blue emission layer, the red emission layer, and the green emission layer.

23. The organic light emitting diode of claim 16, wherein
the capping layer has a thickness of about 200 nanometers or less.

24. The organic light emitting diode of claim 16, wherein
the capping layer has an absorption coefficient of 1.0 or less in the 405 nanometer wavelength.

25. The organic light emitting diode of claim 16, wherein
the capping layer blocks 50% or more of the light of the 405 nanometer wavelength.
